(19) United States
(12) Patent Application Publication
(10) Pub. No.: US 2014/0155334 A1
(43) Pub. Date: Jun. 5, 2014

(54) POLYPHOSPHATE AND PYROPHOSPHATE DERIVATIVE OF SACCHARIDES

(71) Applicants: Jean-Marie Lehn, Strasbourg (FR); Yves Claude Nicolau, Newton, MA (US); Rajamalleswaramma Jogireddy, Strasbourg (FR); Adam H. Brockman, Arlington, MA (US); John Hey, Nashua, NH (US); Yongxin Yu, Nashua, NH (US)

(72) Inventors: Jean-Marie Lehn, Strasbourg (FR); Yves Claude Nicolau, Newton, MA (US); Rajamalleswaramma Jogireddy, Strasbourg (FR); Adam H. Brockman, Arlington, MA (US); John Hey, Nashua, NH (US); Yongxin Yu, Nashua, NH (US)

(73) Assignees: Université de Strasbourg, Strasbourg Cedex (FR); NormOxys, Inc., Brighton, MA (US)

(21) Appl. No.: 14/089,380

(22) Filed: Nov. 25, 2013

Related U.S. Application Data
(63) Continuation of application No. 13/877,094, filed on Nov. 25, 2013.
(60) Provisional application No. 61/388,428, filed on Sep. 30, 2010.

(51) Int. Cl. A61K 31/7028; C07H 11/04

(52) U.S. Cl. CPC A61K 31/7028; C07H 11/04
USPC 514/25; 536/17.1

(57) ABSTRACT

The present invention provides, among other things, phosphorylated and pyrophosphate derivatives of mono-, di- and oligosaccharides, as well as structural derivatives of these compounds. These compounds have a variety of uses including for pharmaceutical applications. Also provided are methods of use in the treatment of disease, including diseases related to oxygen delivery.

### PRIORITY

This application is a 371 application of International Application No. PCT/US2011/054349 filed Sep. 30, 2011, which claims priority to U.S. Provisional Application No. 61/388,428, filed Sep. 30, 2010, both of which are hereby incorporated by reference.

### FIELD OF THE INVENTION

The present invention provides, among other things, polyphosphate and pyrophosphate derivatives of saccharides, as well as structural derivatives of these compounds, for pharmaceutical use.

### BACKGROUND

Numerous diseases, conditions, and disorders, such as, for example, cardiovascular diseases and cancer, involve hypoxia. Thus, active agents that increase oxygen release and/or delivery have substantial pharmaceutical potential.

Some agents may affect oxygen delivery as allosteric effectors of hemoglobin. A key physiological process in the blood aerobic organisms is the delivery of oxygen bound to hemoglobin (Hb) in red blood cells (RBCs) to tissues. Oxygen delivery is regulated, amongst others, by allosteric effectors that bind to hemoglobin and decrease its oxygen binding affinity. One such regulator is 2,3-bisphosphoglycerate (BPG), whose binding to the allosteric pocket of the Hb tetramer has been well characterized. Others substances, such as the natural product myo-inositol hexakisphosphate (IHP) and a variety of polyanionic molecules also act as allosteric Hb effectors. Myo-inositol trispyrophosphate (ITPP), a triple pyrophosphate derivative of IHP, is also able to affect oxygen delivery.

### SUMMARY OF THE INVENTION

The present invention provides polyphosphate or pyrophosphate derivatives of saccharides, such as pyranoses and furanoses, and disaccharides and oligosaccharides containing the same and structural derivatives of these compounds, and pharmaceutical compositions comprising the same. The compounds disclosed herein have biological activity, including for example, as allosteric effectors of hemoglobin and/or regulators of oxygen release or delivery and/or as PI3 kinase inhibitors. The present invention further provides methods for therapy in human or mammalian patients, and methods for synthesis of biologically active compounds and their intermediates.

In one aspect, the invention provides a pharmaceutical composition comprising a polyphosphate or pyrophosphate derivative of a mono-, di- or oligosaccharide. The monosaccharide unit in each case may be a pyranose or a furanose unit. In certain embodiments, the derivatized pyranose or furanose is selected from glucose, mannose, and galactose. In these and other embodiments, the derivatized pyranose or furanose is part of an oligosaccharide (e.g., a disaccharide). In some embodiments, the oligosaccharide is selected from sucrose and lactose, which is derivatized as described herein, including with one or more phosphate or polyphosphate groups.

In another aspect, the invention provides a compound of Formula I:

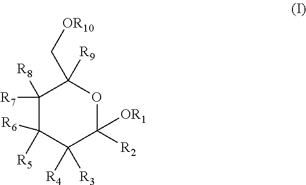

wherein:
R₁ and R₁₀ are independently H, C₁-C₆ alkyl, C₁-C₆ haloalkyl, aryl C₁-C₆ alkyl, phosphate, polyphosphate,

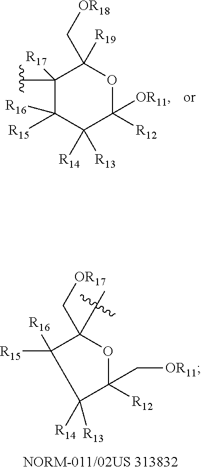

R₂ is H;
R₃, R₄, R₅, R₆, R₇, R₈, R₉, and R₁₀ are independently H, C₁-C₆ alkyl, C₁-C₆ alkenyl, C₁-C₆ alkynyl, C₁-C₆ haloalkyl, aryl C₁-C₆ alkyl, phosphate, or polyphosphate; and
R₁₁, R₁₂, R₁₃, R₁₄, R₁₅, R₁₆, R₁₇, R₁₈, and R₁₉ are independently H, OH, phosphate or polyphosphate;
or a pharmaceutical acceptable salt, stereoisomer, anomer, solvate, and hydrate thereof. In such embodiments, at least one, two, or three of R1 to R10 are phosphate or polyphosphate. In certain embodiments, phosphate groups bound to neighboring positions of the pyranose or sugar ring form an internal pyrophosphate ring. The pyranose may have one or two internal pyrophosphate rings.

In some embodiments, Formula I can alternatively be based on a furanose ring (Formula III). For example, the compound may have the following structure:

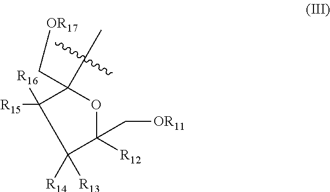

R₁₁, R₁₂, R₁₃, R₁₄, R₁₅, R₁₆, and R₁₇ are independently H, OH, phosphate or polyphosphate;
or a pharmaceutical acceptable salt, stereoisomer, anomer, solvate, and hydrate thereof. In such embodiments, at least one, two, or three of R₁₁ to R₁₇ are phosphate or polyphosphate. In certain embodiments, phosphate groups bound to neighboring positions of the pyranose or sugar ring form an internal pyrophosphate ring. The pyranose may have one or two internal pyrophosphate rings.

In specific embodiments, the compound of Formula I may be any one of the following: 1-O-methyl-α-glucose 2,3,4-trisphosphate (I-1); 1-O-methyl-α-mannose 2,3,4-trisphosphate (I-2); α-glucose 1,2,3,4-tetrakisphosphate (I-3); β-glucose 1,2,3,4-tetrakisphosphate (I-4); α-mannose 1,2,3,4-tetrakisphosphate (I-5); β-mannose 1,2,3,4-tetrakisphosphate (I-6); α-galactose 1,2,3,4-tetrakisphosphate (I-7); β-galactose 1,2,3,4-tetrakisphosphate (I-8); 1-O-methyl-α-glucose tetrakisphosphate (I-9); 1-O-methyl-α-mannose tetrakisphosphate (I-10); α-glucose pentakisphosphate (I-11); α-mannose pentakisphosphate (I-12); α-galactose pentakisphosphate (I-13); lactose octakisphosphate (I-14); and sucrose octakisphosphate (I-15).

In other embodiments, the compounds of Formula I may be stereoisomers which are a D-isomer or L-isomers. In specific embodiments, the compounds of Formula I may be anomers which are in the α or β forms.

In one aspect, the invention provides a compound of Formula II:

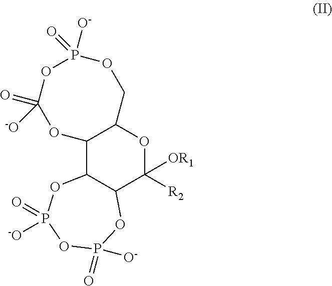

wherein:
R₁ is H, C₁-C₆ alkyl, C₁-C₆ alkenyl, C₁-C₆ alkynyl, C₁-C₆ haloalkyl, aryl C₁-C₆ alkyl, phosphate or polyphosphate; and
R₂ is H;
or a pharmaceutical acceptable salt, stereoisomer, anomer, solvate, and hydrate thereof.

In a specific embodiment, the compound of Formula II may be 1-O-methyl-α-glucose bispyrophosphate (II-1).

In another aspect, the present invention provides a method of treating cancer comprising administering to a subject in need thereof a pharmaceutical composition comprising a therapeutically effective amount of a phosphate, polyphosphate or pyrophosphate derivative of a mono-, di- or oligosaccharide containing at least one pyranose or furanosc unit, or structural mimctics thereof, as described herein.

In yet another aspect, the invention provides a method of treating a cardiovascular disease comprising administering to a subject in need thereof a pharmaceutical composition comprising a therapeutically effective amount of a compound disclosed herein.

In yet another aspect, the invention provides a method of enhancing oxygen delivery to a tissue or organ of a mammal, comprising administering to a subject in need thereof a pharmaceutical composition comprising a therapeutically effective amount of compound disclosed herein.

### DESCRIPTION OF THE FIGURES

FIG. 1 shows the P₅₀ values, Hill coefficients and dissociation constants in stripped human Hb for selected compounds disclosed herein.

FIG. 2 shows the relationship between Hb-oxygen binding (P₅₀) and dissociation constants from Hb (K_{d}) for compounds IHP, ITPP, 1-O-methyl-α-glucose 2,3,4-trisphosphate (8, I-1), 1-O-methyl-α-mannose 2,3,4-trisphosphate (10, I-2), α-glucose 1,2,3,4-tetrakisphosphate (29, I-3), β-glucose 1,2,3,4-tetrakisphosphate (30, I-4), α-mannose 1,2,3,4-tetrakisphosphate (31, I-5), β-mannose 1,2,3,4-tetrakisphosphate (32, I-6), α-galactose 1,2,3,4-tetrakisphosphate (33, I-7), β-galactose 1,2,3,4-tetrakisphosphate (34, I-8), 1-O-methyl-α-glucose tetrakisphosphate (41, I-9), 1-O-methyl-α-mannose tetrakisphosphate (42, I-10), α-glucose pentakisphosphate (47, I-11), α-mannose pentakisphosphate (49, I-12), α-galactose pentakisphosphate (51, I-13), lactose octakisphosphatc (55, I-14), sucrose octakisphosphate (59, I-15) and 1-O-methyl-α-glucose bispyrophosphate (62, II-1). The line corresponds to the linear regression function (R²=0.795) for all compounds studied.

FIG. 3 shows the P₅₀ values for stripped human Hb and corresponding Hill coefficients for compounds BPG, ITPP, IHP, 1-O-methyl-α-glucose 2,3,4-trisphosphate (8, I-1), 1-O-methyl-α-mannose 2,3,4-trisphosphate (10, I-2). All data were extracted from oxygen saturation curves, which were measured in triplicate. Error bars represent the standard deviation.

FIG. 4 shows the P₅₀ values for stripped human Hb and corresponding Hill coefficients for compounds IHP, α-glucose 1,2,3,4-tetrakisphosphate (29, I-3), β-glucose 1,2,3,4-tetrakisphosphate (30, I-4), α-mannose 1,2,3,4-tetrakisphosphate (31, I-5), β-mannose 1,2,3,4-tetrakisphosphate (32, I-6), α-galactose 1,2,3,4-tetrakisphosphate (33, I-7), β-galactose 1,2,3,4-tetrakisphosphate (34, I-8). All data were extracted from oxygen saturation curves, which were measured in triplicate. Error bars represent the standard deviation.

FIG. 5 shows the P₅₀ values for stripped human Hb and corresponding Hill coefficients for compounds IHP, 1-O-methyl-α-glucose tetrakisphosphate (41, I-9), 1-O-methyl-α-mannose tetrakisphosphate (42, I-10) and 1-O-methyl-α-glucose bispyrophosphate (62, II-1). All data were extracted from oxygen saturation curves, which were measured in triplicate. Error bars represent the standard deviation.

FIG. 6 shows the P₅₀ values for stripped human Hb and corresponding Hill coefficients for compounds IHP, α-glucose pentakisphosphate (47, I-11), α-mannose pentakisphosphate (49, I-12) and α-galactose pentakisphosphate (51, I-13). All data were extracted from oxygen saturation curves, which were measured in triplicate. Error bars represent the standard deviation.

FIG. 7 shows the P₅₀ values for stripped human Hb and corresponding Hill coefficients for compounds IHP, lactose octakisphosphate (55, I-14) and sucrose octakisphosphate (59, I-15). All data were extracted from oxygen saturation curves, which were measured in triplicate. Error bars represent the standard deviation.

FIG. 8 shows exemplary compounds having activity against PI3K. The % inhibition of PI3Kα, PI3Kβ, PI3Kγ, PI3Kδ is shown by a scoring system whereby (+++) is the highest inhibitory effect and (−) is the lowest.

### DETAILED DESCRIPTION OF THE INVENTION

The present invention provides, inter alia, phosphate, polyphosphate or pyrophosphate derivatives of mono-, di-, or oligosaccharides containing a pyranose or furanose unit and structural derivatives of these compounds as well as pharmaceutical compositions comprising the same. The compounds and compositions disclosed herein have biological activity as, for example, regulators of oxygen delivery. Accordingly, the present invention further provides methods for therapy in human or mammalian patients in various disease states involving hypoxia, including, for example, cancer and cardiovascular diseases. Also, provided are methods for use in enhancing oxygen delivery and/or PI3 kinase inhibition.

In one aspect, the invention provides a pharmaceutical composition comprising a polyphosphate or pyrophosphate derivative of mono-, di-, or oligosaccharides containing a pyranose or furanose unit. In certain embodiments, the pyranose or furanose is selected from glucose, mannose, and galactose, the pyranose or furanose being derivatized by at least one or two phosphate or polyphosphate groups. In these or other embodiments, the pyranose or furanose is part of a oligosaccharide, such as a disaccharide. In specific embodiments the oligosaccharide comprises from 2 to about 4 monosaccharide units. In some embodiments, the oligosaccharide is selected from sucrose and lactose, with at least one pyranose or furanose unit derivatized as described herein.

Pyranoses are carbohydrates that have a chemical structure that includes a six-membered ring consisting of five carbon atoms and one oxygen atom and that are structurally similar to the oxygen heterocycle pyran. Glucose, a pyranose, is one of the most common of the monosaccharides. In various combinations and permutations, it forms starch, cellulose, sucrose (“table sugar”), and lactose (“milk sugar”), among other things. When metabolized via the glycolytic pathway, it is the major energy source for many living things. Other non-limiting examples of pyranoses include mannose and galactose. Mannose is an important part of the complex sugars, or oligosaccharides, that attach to proteins in the formation of glycoproteins. Galactose combines with glucose to form lactose or “milk sugar.”

Furanoses are carbohydrates that have a chemical structure that includes a five-membered ring consisting of four carbon atoms and one oxygen atom and that are structurally similar to the oxygen heterocycle furan.

In still other embodiments, the pharmaceutical composition comprising a compound that is a phosphate, polyphosphate or pyrophosphate derivative of a pyranose or furanose comprises, collectively, from 2 to about 10 phosphate groups, which may be (independently) in the form of pyrophosphate. In specific embodiments, the compound of the present invention comprises 3, 4, 5, 6, 7, or 8 phosphate groups, which may include pyrophosphate or polyphosphate groups. In other embodiments, the compound comprises multiple pyrophosphate groups. In specific embodiments, the compound comprises 1, 2, 3, 4, 5, 6, 7, or 8 pyrophosphate groups. In some embodiments, at least one or two pyrophosphates are pyrophosphate rings. For example, when positioned off neighboring carbons of the pyranose or furanose ring, two phosphate groups may be condensed to form a pyrophosphate ring.

In another embodiment, the compound comprises one or more derivatized hydroxyls selected from alkoxy (—OR) or acyloxy (—OCOR), where R is selected from alkyl, aryl, acyl, aralkyl, alkenyl, alkynyl, heterocyclyl, polycyclyl, carbocycle, amino, acylamino, amido, alkylthio, carbonyl, sulfonate, alkoxyl, sulfonyl, or sulfoxido, or a salt thereof. In some embodiments, R is alkyl and contains 1 to 10 carbon atoms or in some embodiments, 1, 2, 3, or 4 carbon atoms.

In some embodiments, the pharmaceutical composition is suitable for oral, parenteral, transdermal, topical, intravenous, intraperitoneal, subcutaneous, intramuscular, intradermal, ophthalmic, epidural, intratracheal, sublingual, buccal, rectal, vaginal, nasal or inhalant administration.

In some embodiments, the pharmaceutical composition is in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

In further embodiments, the pharmaceutical composition further comprises an additive selected from an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof.

In certain embodiments, the compound is a pharmaceutically acceptable prodrug or salt thereof, analogous to that which is described, for example, in U.S. Pat. No. 7,618,954, which is hereby incorporated by reference in its entirety. Exemplary salts include a calcium salt, sodium salt, or mixed calcium and sodium salt. Exemplary salts are disclosed in WO 2009/145751, which is hereby incorporated by reference in its entirety. Exemplary salts may include organic cations, alkali metal cations, or alkaline earth cations.

In some embodiments, the dosage regimen utilizing the present compositions may be selected in accordance with a variety of factors including type, species, age, weight, sex and medical condition of the patient; the severity of the condition to be treated; the route of administration; the renal or hepatic function of the patient; and the composition employed. A physician or veterinarian of ordinary skill in the art can readily determine and prescribe the effective amount of the drug required to prevent, counter or arrest the progress of the condition.

Effective dosage amounts of the present invention, when used for the indicated effects, can range from about 25-1000 mg per day. Compositions for in vivo or in vitro use can contain about 20, 50, 75, 100, 150, 250, 500, 750, 1,000, 1,250, 2,500, 3,500, or 5,000 mg of the compound. Appropriate dosages can be determined as set forth in Goodman, L. S.; Gilman, A. The Pharmacological Basis of Therapeutics, 5th ed.; MacMillan: New York, 1975, pp. 201-226. The present compositions can be administered in a single daily dose, or the total daily dosage can be administered in divided doses of two, three or four times daily.

In one aspect, the invention provides a compound of Formula I:

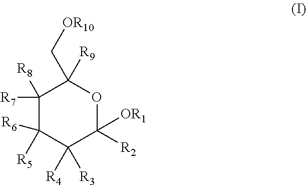

wherein:
R₁ and R₁₀ are independently H, C₁-C₆ alkyl, C₁-C₆ haloalkyl, aryl C₁-C₆alkyl, phosphate, polyphosphate,

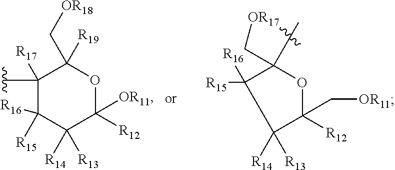

R₂ is H;
R₃, R₄, R₅, R₆, R₇, R₈, R₉, and R₁₀ are independently H, C₁-C₆ alkyl, C₁-C₆ alkenyl, C₁-C₆ alkynyl, C₁-C₆ haloalkyl, aryl C₁-C₆ alkyl, phosphate, or polyphosphate; and
R₁₁, R₁₂, R₁₃, R₁₄, R₁₅, R₁₆, R₁₇, R₁₈, and R₁₉ are independently H, OH, phosphate, or polyphosphate;
or a pharmaceutical acceptable salt, stereoisomer, anomer, solvate, and hydrate thereof. In such embodiments, at least one, two, or three of R1 to R10 are phosphate or polyphosphate. In certain embodiments, phosphate groups bound to neighboring positions of the pyranose (directly or indirectly) or sugar ring form an internal pyrophosphate ring. The pyranose may have one or two internal pyrophosphate rings.

Alternatively, the structure is Formula III as follows:

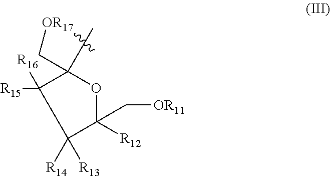

R₁₁, R₁₂, R₁₃, R₁₄, R₁₅, R₁₆, and R₁₇ are independently H, OH, phosphate, or polyphosphate;
or a pharmaceutical acceptable salt, stereoisomer, anomer, solvate, and hydrate thereof. In such embodiments, at least one, two, or three of R₁₁ to R₁₇ are phosphate or polyphosphate. In certain embodiments, phosphate groups bound to neighboring positions of the pyranose (directly or indirectly) or sugar ring form an internal pyrophosphate ring. The pyranose may have one or two internal pyrophosphate rings.

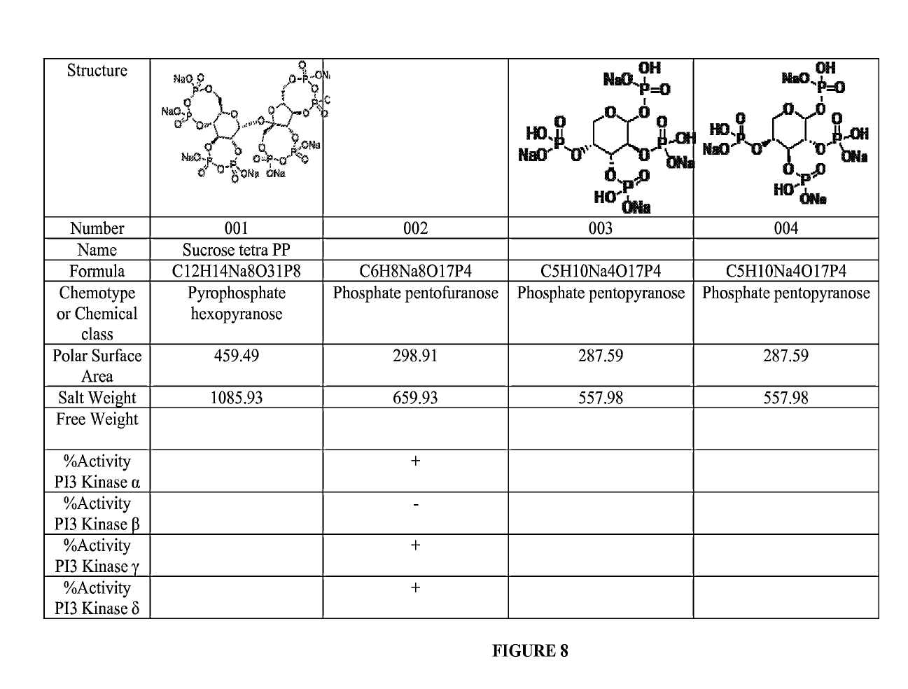

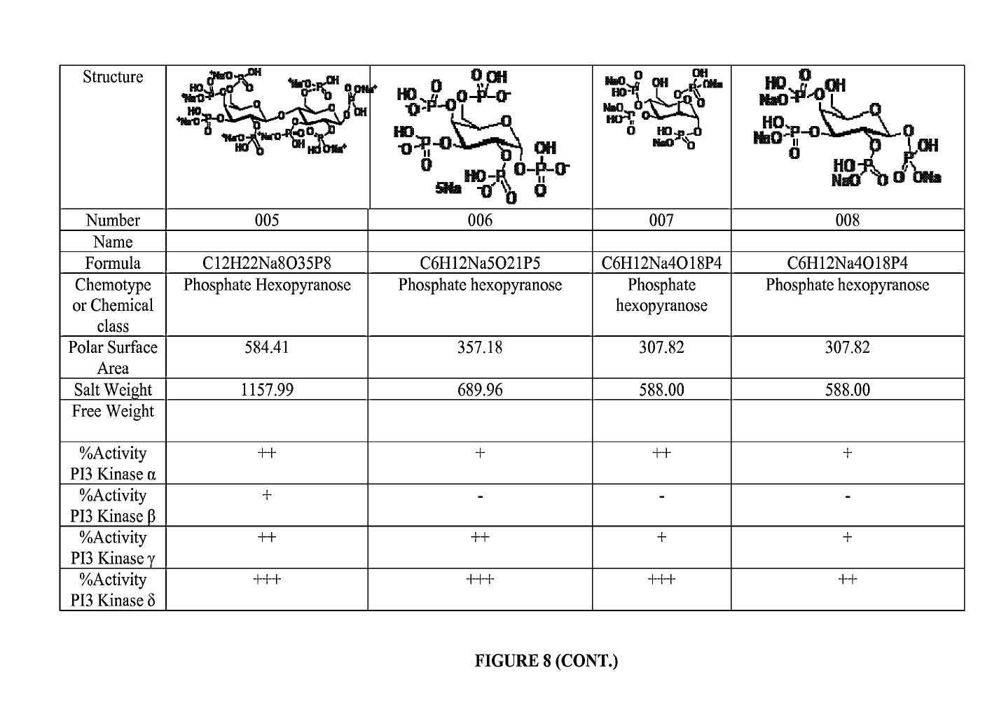

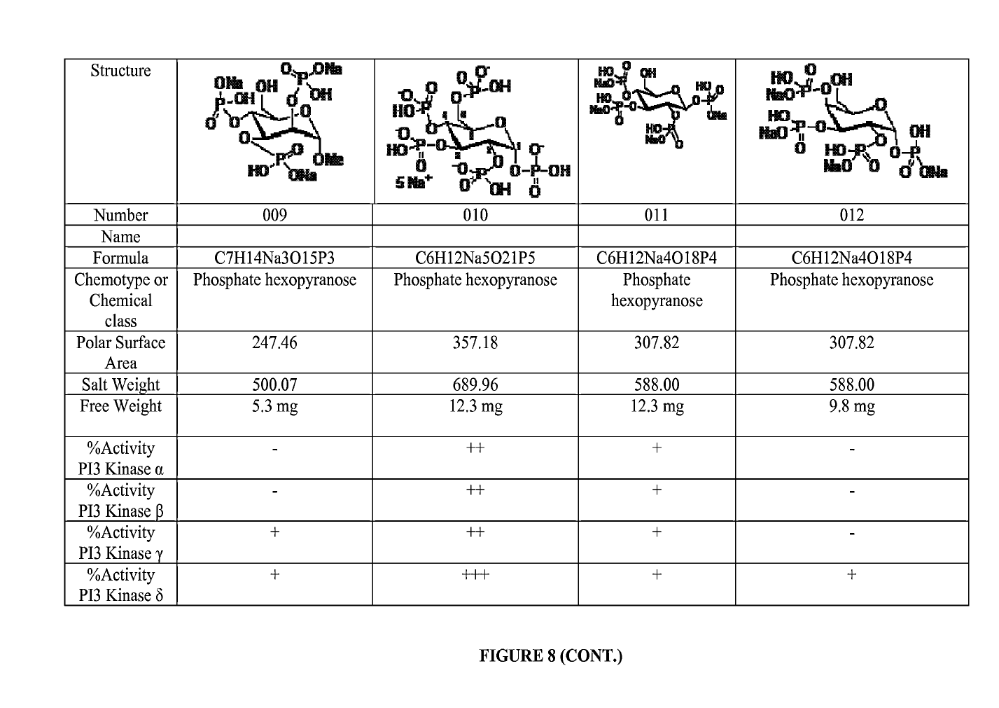

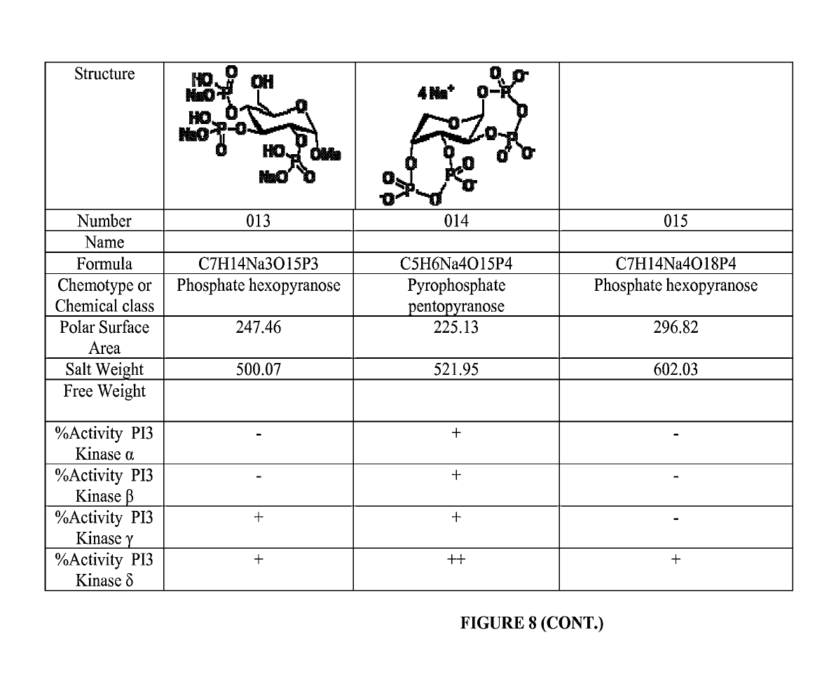

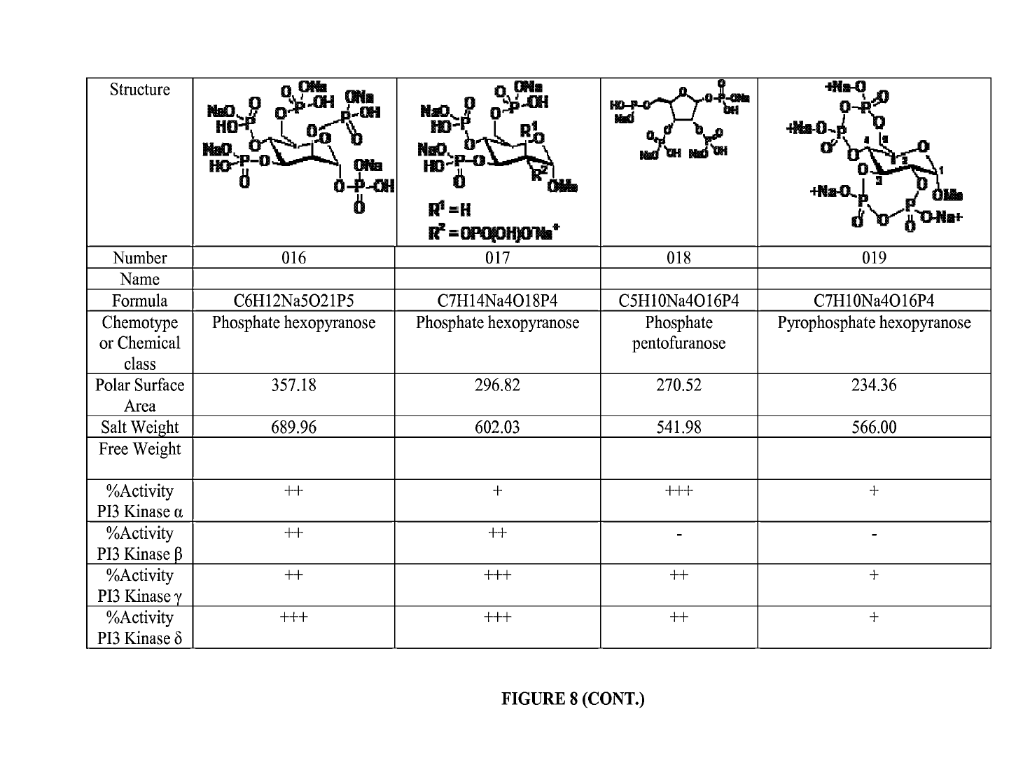

In one embodiment, the compound of Formula III is 018 (FIG. 8)

In some embodiments, the compounds of Formula I have at least the R₄, R₆, and R₈ as phosphate. Alternatively or in addition, when R₅, R₇, R₉, and R₁₀ are H and R₁ is methyl, the hemiacetal carbon is not in the α-anomeric form.

In some embodiments, the compounds of Formula I is one of:

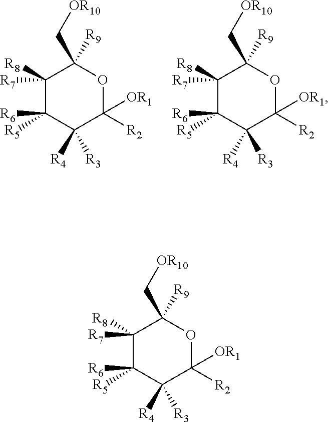

wherein such compounds have a phosphate or polyphosphate group at positions R₃, R₆, and R₇, or a pharmaceutical acceptable salt, stereoisomer, anomer, solvate, and hydrate thereof.

In a specific embodiment, any of the above compounds have an R₁₀ which is H and a R₁ which is methyl. In another specific embodiment, any of the above compounds have an R₁₀ which is phosphate or polyphosphate. In another specific embodiment, any of the above compounds have an R₁ which is phosphate or polyphosphate. In another specific embodiment, any of the above compounds have an R₁ and R₁₀ which are phosphate or polyphosphate. In each embodiment, polyphosphate may be pyrophosphate.

In specific embodiments, the compound of Formula I may be any one of the following: 1-O-methyl-α-glucose 2,3,4-trisphosphate (I-1); 1-O-methyl-α-mannose 2,3,4-trisphosphate (I-2); α-glucose 1,2,3,4-tetrakisphosphate (I-3); β-glucose 1,2,3,4-tetrakisphosphate (I-4); α-mannose 1,2,3,4-tetrakisphosphate (I-5); β-mannose 1,2,3,4-tetrakisphosphate (I-6); α-galactose 1,2,3,4-tetrakisphosphate (I-7); β-galactose 1,2,3,4-tetrakisphosphate (I-8); 1-O-methyl-α-glucose tetrakisphosphate (I-9); 1-O-methyl-α-mannose tetrakisphosphate (I-10); α-glucose pentakisphosphate (I-11); α-mannose pentakisphosphate (I-12); α-galactose pentakisphosphate (I-13); lactose octakisphosphate (I-14); and sucrose octakisphosphate (I-15).

In other embodiments, the compounds of Formula I may be stereoisomers which are a D-isomer or L-isomers. In specific embodiments, the compounds of Formula I may be anomers which are in the α or β forms.

In another aspect, the invention provides a compound of Formula II:

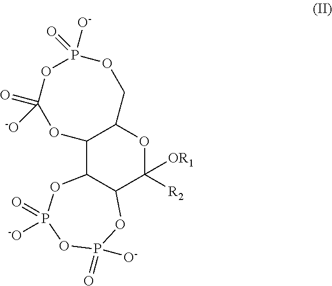

wherein:
R₁ is H, C₁-C₆ alkyl, C₁-C₆ alkenyl, C₁-C₆ alkynyl, C₁-C₆ haloalkyl, aryl C₁-C₆ alkyl, phosphate or polyphosphate; and
R₂ is H;
or a pharmaceutical acceptable salt, stereoisomer, anomer, solvate, and hydrate thereof.

In specific embodiments, the compound of Formula II may be 1-O-methyl-α-glucose bispyrophosphate (II-1).

The descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge. Standard methods for the preparation of pharmaceutically acceptable salts and their formulations are well known in the art, and are disclosed in various references, including for example, “Remington: The Science and Practice of Pharmacy,” A. Gennaro, ed., 20th edition, Lippincott, Williams & Wilkins, Philadelphia, Pa.

“Solvate”, as used herein, means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate”. Examples of hydrate include, but are not limited to, hemihydrate, monohydrate, dihydrate, trihydrate, hexahydrate, etc. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

The term “prodrug” refers to a precursor of a pharmaceutically active compound wherein the precursor itself may or may not be pharmaceutically active but, upon administration, will be converted, either metabolically or otherwise, into the pharmaceutically active compound or drug of interest. For example, prodrug can be an ester, ether, or amide form of a pharmaceutically active compound. Various types of prodrug have been prepared and disclosed for a variety of pharmaceuticals. See, e.g., Bundgaard, H. and Moss, J., J. Pharm. Sci. 78: 122-126 (1989).

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Excipient” refers to a diluent, adjuvant, vehicle, or carrier with which a compound is administered.

In another aspect, the present invention provides a method of treating cancer comprising administering to a subject in need thereof a pharmaceutical composition comprising a therapeutically effective amount of a polyphosphate or pyrophosphate derivative of a mono-, di- or oligosaccharide containing a pyranose or furanose unit or structural mimetics thereof described herein. In some embodiments, the pharmaceutical composition administered is a compound of Formulae I, II, III. In specific embodiments, the cancer to be treated is a breast cancer, prostate cancer, renal cell cancer, brain cancer, ovarian cancer, colon cancer, bladder cancer, pancreatic cancer, stomach cancer, esophageal cancer, cutaneous melanoma, liver cancer, lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, lip and oral cancer, skin cancer, leukemia or multiple myeloma.

In another embodiment, the treatment of cancer further comprises administering to the subject a therapeutically effective amount of a chemotherapeutic agent. Since chemotherapeutic agents can lose effectiveness against hypoxic tumors, the compounds of the instant invention may provide for synergy with chemotherapeutic agents. Such therapeutic agents can include, for example, amino glutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

In still other embodiments, one or more compounds of the invention are administered together with radiation therapy. Radiation therapy often is of limited effectiveness for hypoxic tumors.

In yet another aspect, the invention provides a method of treating a cardiovascular disease comprising administering to a subject in need thereof a pharmaceutical composition comprising a therapeutically effective amount of a polyphosphate or pyrophosphate derivative of a mono-, di- or oligosaccharide containing a pyranose or furanose unit or structural mimetics thereof. In some embodiments, the pharmaceutical composition administered is a compound of Formulae I, II, III. In some embodiments, the cardiovascular disease is a coronary infarction, a pulmonary disease, congestive heart failure, a myocardial infarction, a peripheral vascular disease, stroke, an intermittent claudication, or arteriosclerosis. In a specific embodiment, the cardiovascular disease is congestive heart failure.

In still another aspect the invention provides a method of enhancing oxygen delivery to a tissue or organ of a mammal, comprising administering to a subject in need thereof a pharmaceutical composition comprising a therapeutically effective amount of a therapeutically effective amount of a polyphosphate or pyrophosphate derivative of a mono-, di- or oligosaccharide containing a pyranose or furanose unit or structural mimetics thereof (as described herein). In some embodiments, the pharmaceutical composition administered is a compound of Formulae I, II, III.

In certain embodiments, the compounds may act as allosteric effectors of hemoglobin, to enhance the delivery of oxygen to tissues. For example, where the condition is cancer, oxygenation of the tumor may result in increased sensitivity to radiation or increased chemosensitivity, or may reduce the angiogenic and/or metastatic potential of the tumor. These embodiments for allosteric effectors of hemoglobin are described in one or more of U.S. Pat. No. 7,745,423, U.S. Pat. No. 7,618,954, and U.S. 2008/0200437, each of which are hereby incorporated by reference in their entirety. Where the condition is heart failure, such as congestive heart failure, the compound may increase the efficiency of oxygen delivery to body tissues, including the heart, to thereby ameliorate or slow progression of the disease, as described in U.S. Pat. No. 7,618,954, which is hereby incorporated by reference in their entirety. Additional conditions, for which the allosteric effectors of hemoglobin find use, include anemia, hypoxia, and Alzheimer's disease.

Furthermore, the present compounds may act as kinase inhibitors, including, by way of non-limiting example, inhibitors of PI3K, including a class I PI3K, class II PI3K, class III, and/or class IV PI3K as described in U.S. Ser. No. 61/486,001, which is hereby incorporated by reference in its entirety.

In certain embodiments of any of the above methods, the administration of the pharmaceutical composition is oral, parenteral, transdermal, topical, intravenous, intraperitoneal, subcutaneous, intramuscular, intradermal, ophthalmic, epidural, intratracheal, sublingual, buccal, rectal, vaginal, nasal or inhalant. In other certain embodiments of any of the above methods, the pharmaceutical composition is administered in a composition comprising an additive selected from an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof. In other certain embodiments of any of the above methods, the pharmaceutical composition is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

### EXAMPLES

### Example 1

### Synthesis of Compounds

General Experimental Methods

All chemicals were purchased from Sigma, Aldrich or Fluka and were used without further purification. The resins Dowex 50WX8-200 and Marathon C Na⁺ were purchased from Sigma-Aldrich and washed with distilled water before use. ¹H, ¹³C, and ³¹P NMR spectra were recorded with a Bruker AC-400 spectrometer. Mass spectra were determined by the Service Commun de Spectrométrie de Masse (Institut d'Ingénierie Supramoléculaire). ITPP (myo-inositol trispyrophosphate hexasodium salt) was manufactured by Carbogen AMCIS (Switzerland), following improved synthetic procedures, derived from those previously described. BPG (2,3-bisphospho-D-glyceric acid pentasodium salt) was purchased from Sigma (USA) and IHP (myo-inositol hexakisphosphate) was purchased from Sigma-Aldrich (Italy).

General Procedure for the Phosphorylation Reactions:

In a solution of the carbohydrate (1 mmol) in DMF (20 mL), a 0.45 M solution of tetrazole in acetonitrile (2.25 eq for each hydroxyl group) and dibenzyl N,N-diisopropylphosphoramidite (1.5 eq for each hydroxyl group) were added together under an argon atmosphere at room temperature. The resulting slurry was vigorously stirred at room temperature for 24 h. The mixture was then cooled to −40° C. and a solution of 70% mCPBA (1.75 eq for each hydroxyl group) in CH₂Cl₂ (1.5 mL per mmol mCPBA) was added dropwise and the mixture was left to stir for a total of 12 h while it was allowed to warm up to room temperature. The mixture was subsequently diluted with CH₂Cl₂ (150 mL per mmol of starting material) and washed with a 10% aqueous solution of sodium sulfite (2×10 mL per mmol mCPBA), a saturated aqueous solution of sodium bicarbonate (2×10 mL per mmol mCPBA), H₂O (5 mL per mmol mCPBA), and saturated brine (5 mL per mmol mCPBA). The organic phase was dried (MgSO₄) and the solvents were removed under reduced pressure. The obtained residue was purified by flash column chromatography. DMF was used for the naked sugars. For 6-OTBDPS protected sugars, the tetrazole solution in MeCN was directly poured into the flask containing the carbohydrate derivative and thus MeCN was used as a solvent for the sugar as well.

General Procedure for the Hydrogenation Reactions—Preparation of Triethylammonium Salts:

Benzyl phosphate (1 mmol) was dissolved in a 1:1 mixture of ethanol and H₂O (60 mL). Triethylamine (5 eq for each phosphate) was added to the resulting emulsion followed by 10% Pd/C (0.3 g for each phosphate). This mixture was left to vigorously stir under a H₂ atmosphere (1 Atm) or shaked in a high pressure hydrogenator (3 Atm) at room temperature for 24 h. The catalyst was removed by filtration through an LCR/PTFE hydrophilic membrane (0.5 μm), and the filtrate was washed with a 1:1 mixture of ethanol and H₂O (2×50 mL for each mmol of starting material). The combined filtrates were evaporated under reduced pressure (60° C.) and the obtained residue was dried under high vacuum to give the corresponding triethylammonium salt.

General Procedure for the Hydrogenation Reactions—Direct Preparation of Sodium Salts:

The above described procedure was used for the preparation of sodium salts but, instead of triethylamine, NaHCO₃ (1 eq for each phosphate) was used.

General Procedure for the Synthesis of Sodium Phosphates and Pyrophosphates from the Triethylammonium Phosphates:

A solution of ammonium salt of phosphate (1 mmol) in H₂O (10 mL) was passed through a column containing Dowex H⁺ and eluted with distilled water until all the acidic fractions were collected. Acidic fractions were then poured into a flask containing Dowex Na⁺ (50 g for both resins of the same capacity). The mixture was stirred for 30 min, filtered off through a sintered funnel, washed twice with 30 mL of distilled water, and the clear solution was evaporated to dryness. Alternatively, the pH of the acidic fractions was adjusted to neutral upon titration with a NaOH solution. For some ammonium salts a single direct passing through a Dowex Na⁺ column was enough for the exchange of the counter cations.

General Procedure for Silylation Reactions:

The desired carbohydrate (1 mmol) was dissolved in dry DMF (20 mL) and cooled to 0° C. under argon. Et₃N (1.4 eq) and DMAP (10 mg) were added, followed by the slow addition (2 h) of TBDPSCl (1 mmol). The reaction mixture was allowed to warm up to room temperature and left stirring for 24 h. Ethyl acetate (50 mL) was added and the mixture was washed with H₂O (2×50 mL) and saturated brine (2×50 mL). The organic layer was dried (Na₂SO₄), the solvents were removed under reduced pressure, and the obtained residue was purified with column chromatography.

General Procedure for the Desilylation Reactions:

The silylated compound (1 mmol) was dissolved in THF (40 mL) and the mixture was cooled to 0° C. Subsequently, a mixture of TBAF (5 mmol, 1 M in THF) and AcOH (5 mmol) dissolved in ice cold THF (20 mL) was added dropwise under argon in a period of 1 hour. The reaction mixture was allowed to warm to room temperature and stirred for 5-12 h (checked by TLC). Ethyl acetate (50 mL) was added and the organic phases were washed with H₂O (50 mL) and saturated brine (50 mL). The organic phase was dried (MgSO₄) and the solvents were removed under reduced pressure. The obtained residue was purified by flash column chromatography.

General Procedure for the Formation of Pyrophosphates:

The triethyl ammonium salt of the carbohydrate phosphate (1 mmol) was dissolved in a mixture of acetonitrile/water in a ratio of 2:1 (45 mL), or in neat acetonitrile (30 mL) and N,N-dicyclohexyl carbodiimide (1 equiv for each phosphate) was added in one portion. The mixture was stirred under reflux for 18-24 h, and then cooled down and concentrated under vacuum. H₂O (2×30 mL) was added and the N,N-dicyclohexyl urea was filtered off through a sintered funnel. The filtrate was concentrated under vacuum to give the pure triethylammonium salt of the pyrophosphate.

General Strategy for Synthesis of Polyphosphate and Cyclic Pyrophosphate Derivatives of Hexopyranoses

Previous X-ray crystallographic analysis of IHP has shown that it does not bind the allosteric pocket of Hb with all its phosphates. See D. A. Waller, R. C. Liddington, Acta Crystallogr. B, 1990, 46, 409. Rather, only three are properly orientated for this purpose. Knowing this, the present synthetic plans were undertaken, in part, to evaluate and exploit features of the molecular recognition of effectors in the allosteric pocket of Hb, e.g. the effect of the number of phosphates, the most appropriate conformation for binding (for instance, mannose and galactose with one axial OH group are closer related to IHP) and the role of the anomeric phosphate (α- or β-), which is the most chemically labile. In light of this, some of the compounds described herein have double protection of positions 1- and 6- of monosaccharides. Accordingly, some of the compounds described herein are corresponding tris phosphorylated derivatives. Further, proper selective unmasking of position 1- or 6- of hexoses allowed the synthesis of tetrakis phosphorylated derivatives and the possible simultaneous formation of two cyclic PPs in a row. In addition, protection of position 6-allowed for the synthesis of both α- and β-anomers of the tetrakis phosphorylated derivatives. Finally, perphospharylation of naked monosaccharides and disaccharides allowed for the synthesis of pentakis and octakis phosphates, respectively. While the invention is not to be limited as such, the examples described herein employ selected monosaccharides (i.e., glucose, mannose, and galactose) and selected disaccharides (i.e., a reducing disaccharide, lactose and a non-reducing disaccharide, sucrose).

Synthesis of Tris Phosphorylated Monosaccharides

Scheme I shows the synthesis of the tris phosphorylated derivatives 8 (I-1) and 10 (I-2) of glucose and mannose respectively, from their silylated methyl glycoside precursors 1 and 2. The reagents and conditions used, with reference to Scheme I, included: a) 1) (BnO)₂PN(iPr)₂, tetrazole, MeCN, RT; 2) mCPBA, CH₂Cl2, −40° C. to RT; b) TBAF, AcOH, THF, 0° C.; c) H₂ (1 Atm), Pd/C, Et₃N, EtOH/H₂O (1:1), RT; d) Dowex H⁺, H₂O then Dowex Na⁺, H₂O. [DBP=P(O)(OBn)₂].

The sodium salts of the tris phosphorylated glucose and mannose derivatives 8 (I-1) and 10 (I-2) were prepared from the known 6-O-t-butyldiphenylsilyl (TBDPS) glucose and mannose methyl glycosides (1 and 2, respectively). Compounds 1 and 2 were individually subjected to a phosphorylation reaction using dibenzyl N,N-diisopropylphosphoramidate and tetrazole in dry MeCN, under argon at room temperature (RT) for 24 h. The initially formed phosphites were directly oxidized with m-chloro-perbenzoic acid (mCPBA) to give compounds 3 and 4 in 76 and 73% yield for glucose and mannose, respectively. Removal of the TBDPS protecting group was achieved using a buffered tetrabutylammonium fluoride (TBAF) solution at 0° C. and yielded compounds 5 and 6 (84% in both cases).

The benzyl esters 5 and 6 were deprotected upon catalytic hydrogenolysis (H₂ in the presence of Pd/C and triethylamine) to give the triethylammonium salts 7 and 9. These were transformed to sodium salts 8 (I-1) and 10 (I-2) using a sequence of cation exchange columns first in H⁺ and subsequently in Na⁺ forms.

In general, the triethylammonium salts were required for the preparation of the corresponding PPs, but the Na⁺ salts could be also directly obtained by performing the hydrogenation reaction in the presence of NaHCO₃. In the direct formation of Na⁺ salts it is noted that the gummy starting material was carefully dried and weighed, since an exact amount of NaHCO₃ is required (one equivalent per phosphate) in order to avoid contamination of the final product. In contrast, an excess of base (Et₃N) was easily removed under vacuum when the corresponding triethylammonium salts were prepared. Transformation of the Et₃NH⁺ salt into the H⁺ and then Na⁺ forms using ion exchange procedures provided an indirect and safe way to obtain the sodium salts.

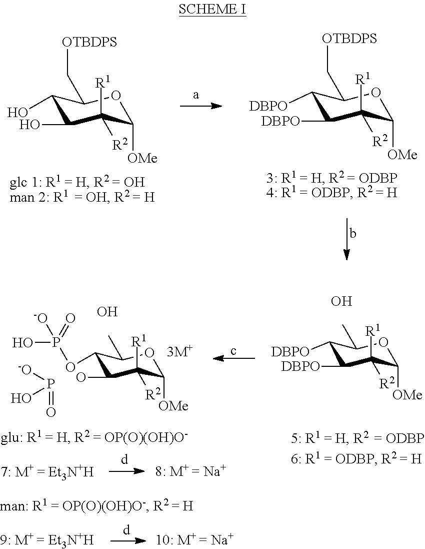

Synthesis of Tetrakis Phosphorylated Monosaccharides

Scheme II shows the synthesis of the tetrakis phosphorylated derivatives 29-34 (I-3 to I-8) of glucose, mannose and galactose from the silylated precursors 14-16. The reagents and conditions used, with reference to Scheme II, included: a) TBDPSCl, Et₃N, DMAP, DMF, 0° C. to RT; b) 1) (BnO)₂PN(iPr)₂, tetrazole, MeCN, RT; 2) mCPBA, CH₂Cl₂, −40° C. to RT; c) TBAF, AcOH, THF, 0° C.; d) H₂ (1 Atm), Pd/C, NaHCO₃, EtOH/H₂O (1:1), RT. [DBP=P(O)(OBn)₂].

Reaction of parent sugars with TBDPS chloride was used to selectively block the primary hydroxyl group at position 6. Phosphorylation of these 6-O-silylated precursors proceeded smoothly in MeCN and with a similar yield of about 80% in each case. Both anomers were formed for all three sugars, although in different proportions. Glucose and mannose gave α- and β-anomers (17/18 and 19/20, respectively) in a ratio of around 5:3, whereas the opposite ratio, around 3:5, was observed for the galactose anomers (21, 22). Glucose and mannose anomers were separated by column chromatography. The galactose derivatives were practically inseparable in large scale and were taken forward as a mixture; nevertheless, a small amount of each anomer was isolated for characterization. Removal of the TBDPS protecting group was performed under carefully controlled conditions (0° C., near neutral pH) in order to prevent losing the sensitive and labile anomeric phosphate (yields 61-81%, compounds 23-28). At this stage galactose anomers were also separated. Finally, hydrogenation in the presence of NaHCO₃ provided directly the sodium salts of both anomers of all monosaccharides (29-34 (I-3 to I-8)) in excellent yields (>99%). No phosphate migration was observed for all of these derivatives.

The assignment of the α- and β-anomers for the glucose and galactose derivatives was possible, in view of the difference in coupling constant of the two vicinal protons at positions 1 and 2. A doublet of doublets was always present in the spectra of all α-anomers (17, 23, 29 (I-3) and 21, 27, 33 (I-7)) with a small coupling constant indicating an eq/ax relative conformation of protons at positions 1 and 2 (³J_{H1,H2}=2.6-3.4 Hz), and a larger one due to coupling of proton H1 with the neighboring phosphorous nucleus (³J_{H1,P}=5.4-7.1 Hz). In contrast, for the β-anomers (18, 24, 30 (I-4) and 22, 28, 34 (I-8)), a triplet was observed, due to similar large values for the coupling of vicinal protons and for the heteronucleus coupling (³J_{H1,H2}≈³J_{H1,P}=6.5-7.9 Hz) indicating, thus, an ax/ax orientation of the protons.

For the mannose derivatives, however, both eq/eq and ax/eq couples of protons gave smaller coupling constants. Therefore, the assignment of α- and β-anomers was based on the comparison of the chemical shifts with those presented in the literature for α- and β-1-monophosphorylated mannose derivatives, which show considerable differences in both ¹H and ¹³C NMR spectra. According to these data, for hexopyranonses like mannose which, based on the NMR spectra seems to present the ⁴C₁ conformation, the anomeric proton signal of the α-anomer appears at lower field than that of the β-form. See S. J. Angyal, Angew. Chem. Int. Ed. 1969, 8, 157. S. J. Angyal, Angew. Chem. Int. Ed. 1969, 8, 157. This was the case, as expected, for all glucose and galactose derivatives, as well. Shifts for H-5 of mannose α-anomers are found downfield in comparison to the corresponding shifts of the β-isomers. Moreover, the ¹³C NMR data display downfield shifts for C-3 and C-5 for β-phosphates. These characteristic features apply also for derivatives, both protected phosphorylated (19, 20, 25, 26) and sodium salts (31, 32). The data obtained for the perphosphorylated mannose derivatives 44, 48 and 49 provide further evidence for the α-orientation of these carbohydrates (Scheme IV infra).

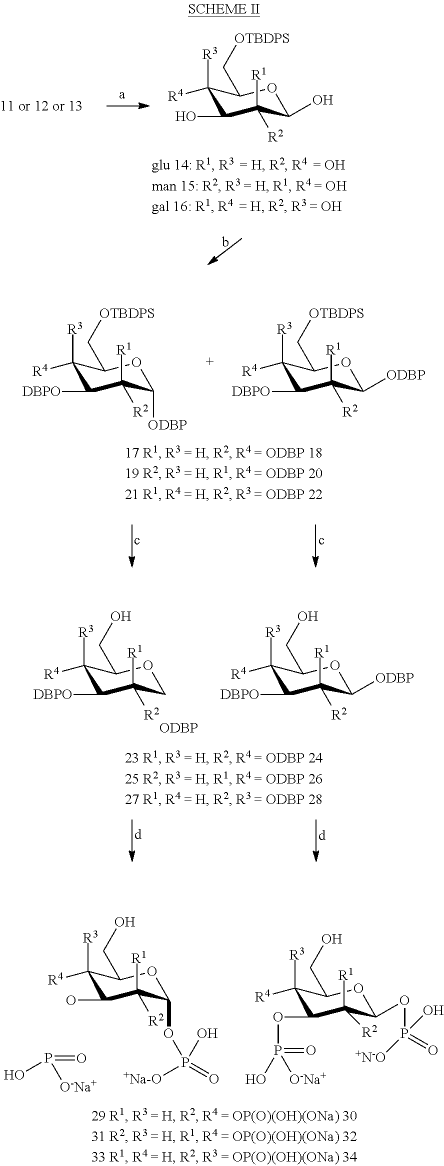

Scheme III shows the synthesis of the tetrakis phosphorylated derivatives of glucose 41 (I-9) and mannose 42 (I-10) from the corresponding methyl glycosides 35 and 36. The reagents and conditions used, with reference to Scheme III, included: a) 1) (BnO)₂PN(iPr)₂, tetrazole, DMF, MeCN, RT; 2) mCPBA, CH₂Cl₂, −40° C. to RT; b) H₂ (1 Atm), Pd/C, Et₃N, EtOH/H₂O (1:1), RT; c) Dowex H⁺, H₂O then Dowex Na⁺, H₂O. [DBP=P(O)(OBn)₂].

To synthesize 2,3,4,6-tetrakis phosphorylated glucose and mannose derivatives, the commercially available glycosides 35 and 36 were used. The free hydroxyl groups in 35 and 36 were all simultaneously phosphorylated (compounds 37 and 38) under the standard protocol stated supra (in 94% and 79% yields, respectively) to give, via the triethylammonium salts 39 and 40, the final sodium salts 41 (I-9) and 42 (I-10) in excellent overall yields. These glucose and mannose derivatives, had four phosphates in a row and the remaining anomeric hydroxyl group protected as methyl ethers.

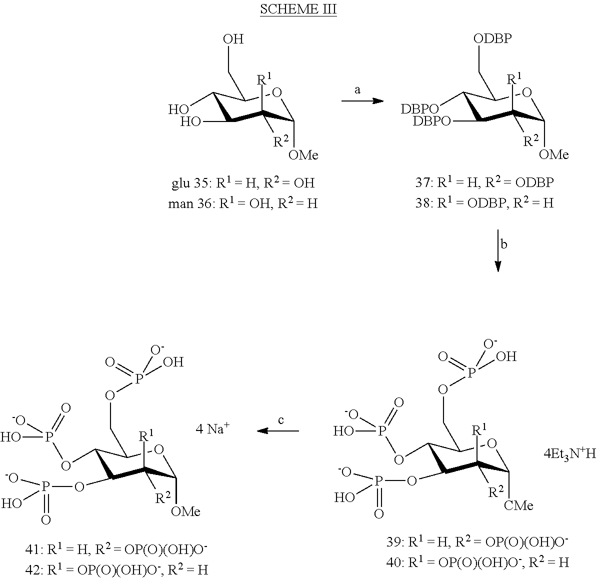

Synthesis of Pentakis Phosphorylated Monosaccharides

Scheme IV shows the synthesis of the pentakisphosphorylated derivatives 46-51 of glucose (11), mannose (12) and galactose (13). The reagents and conditions used, with reference to Scheme IV, included: a) 1) (BnO)₂PN(iPr)₂, tetrazole, DMF, MeCN, RT; 2) mCPBA, CH₂Cl₂, −40° C. to RT; b) H₂ (1 Atm), Pd/C, Et₃N, EtOH/H₂O (1:1), RT; c) Dowex H⁺, H₂O then Dowex Na⁺, H₂O. [DBP=P(O)(OBn)₂].

Glucose (11), mannose (12) and galactose (13) were independently subjected to a phosphorylation reaction using dibenzyl N,N-diisopropylphosphoramidate and tetrazole in dry DMF/MeCN, under argon at RT for 24 h. The initially formed phosphites were directly oxidized with mCPBA to give compounds 43, 44 and 45 in 55%, 62% and 65% yield, respectively. The benzyl esters were deprotected upon catalytic hydrogenolysis (H₂ in the presence of Pd/C) to give the Et₃NH⁺ salts 46, 48 and 50 in very good yields (>94%). The latter derivatives were then transformed into the sodium salts 47 (I-11), 49 (I-12), and 51 (I-13) applying a sequential ion exchange with Dowex H⁺ and subsequently Dowex Na⁺ resins in quantitative yields.

In the cases of the perphosphorylated monosaccharides there was a dramatic change regarding the selectivity of the anomeric positions. In contrast to the 1,2,3,4-tetrakis phosphorylated derivatives, only one anomer was formed for the pentakis phosphorylated analogues. Without wishing to be bound by theory, the bulkiness of the TBDPS protective group in conjunction with the effect of solvent used (MeCN instead of DMF/MeCN), may have influenced this anomeric effect and lead to the formation of both anomers. It is well known that the equilibrium compositions of sugars in solution are affected by temperature, the nature of the solvent, and the presence of substituents. If the solvent is less polar than water, the increased anomeric effect is predicted to, favor the α-pyranose over the β-pyranose form when the sugar is in the ⁴C₁ conformation. However, other, unexpected changes in the anomeric composition are also observed when the solvent or the substituent are altered. See Angyal, supra.

The α-orientation for glucose and galactose derivatives was indicated by the proton ¹H NMR spectra where the signals of the anomeric protons appear as doublet of doublets with a coupling constant corresponding to an eq/ax relative conformation of protons at positions 1 and 2 (³J_{H1,H2}) from 3.0 to 3.4 Hz. The coupling constant of the anomeric proton with the neighboring phosphorous nucleus (³J_{H1,P}) was in the range of 6.0 to 7.4 Hz. The coupling constants of compounds (43, 46 47 (I-11) and 45, 50, 51 (I-13)) were quite similar to those of the α-anomers of the tetrakis phosphorylated derivatives (17, 23, 29 and 21, 27, 33) shown in Scheme II, and in accordance with the data reported in the literature for α-1-phosphorylated carbohydrates, where substituents on position 2 are equatorially oriented. For the mannose derivatives 44, 48 and 49 the comparison of their ¹H and ¹³C-NMR spectra with those of the 1,2,3,4-tetrakis phosphorylated derivatives indicated an α-orientation.

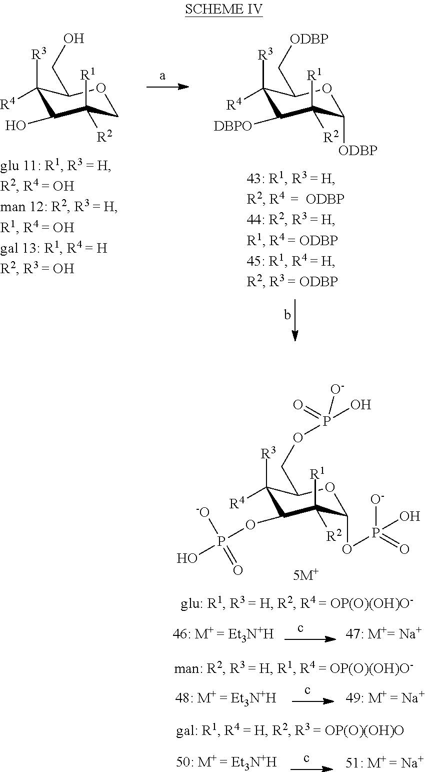

Synthesis of Octakis Phosphorylated Disaccharides

Scheme V shows the synthesis of perphosphorylated derivatives 54 and 55 (I-14) of lactose 52. The reagents and conditions used, with reference to Scheme V, included: a) 1) (BnO)₂PN(iPr)₂, tetrazole, DMF, MeCN, RT; 2) mCPBA, CH₂Cl₂, −40° C. to RT; 1)) H₂ (3 Atm), Pd/C, Et₃N, EtOH/H₂O (1:1), RT; c) Dowex H⁺, H₂O then Dowex Na⁺, H₂O. Lactose (52), a reducing disaccharide, was subjected to the same sequence of reactions (phosphorylation, hydrogenation and ion exchange) as the monosaccharides supra, to give the perphosphorylated lactose derivatives 53 (in a 1:4 ratio of α- and β-anomers). A small quantity of pure β-53 was obtained, by column chromatography, whereas the rest remained as a mixture with the α-isomer.

The anomeric ratio of lactose derivatives was determined based on their ¹H NMR spectra. Although it was relatively easy to make the assignment of the anomeric proton of the minor isomer of 53, it was practically impossible to observe the anomeric proton for the major one (obscured by the methylene protons of benzyl groups). Therefore, it proved much easier to assign the α- and β-anomer in the proton NMR spectra of sodium salts 55 (I-14). The minor anomer gave a signal at 5.57 ppm in the form of a doublet of doublets, ³J_{H1,H2}=3.5 Hz and ³J_{H1,P}=7.1 Hz. For the major lactose derivative 55 (I-14) a triplet appeared at 5.02 ppm for the anomeric proton, (³J_{H1,H2}=³J_{H1,P}=8.0 Hz), indicating an ax/ax orientation of the protons. The same distribution of anomers could be easily assigned for the lactose derivatives 53 and 54. Since all perphosphorylation reactions were performed in the same solvent system, it was predicted that lactose gave a mixture of anomers, with the β-one predominating, due solely to steric factors, whereas the preference for the formation of α-anomers for glucose mannose and galactose derivatives (43, 44, 45, respectively) appeared to result from the anomeric effect.

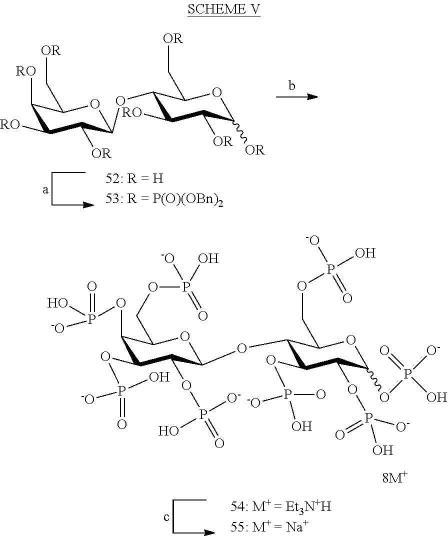

Scheme VI shows the synthesis of the perphosphorylated derivatives 58 and 59 of sucrose 56. The reagents and conditions used, with reference to Scheme VI, included: a) 1) (BnO)₂PN(iPr)₂, tetrazole, DMF, MeCN, RT; 2) mCPBA, CH₂Cl₂, −40° C. to RT; H₂ (3 Atm), Pd/C, Et₃N, EtOH/H₂O (1:1), RT; c) Dowex H⁺, H₂O then Dowex Na⁺, H₂O, Sucrose (56), a non reducing disaccharide, was subjected to a phosphorylation reaction to give compound 57 in 77% yield. This benzyl ester was deprotected upon catalytic hydrogenolysis to generate the triethylammonium salt 58 in very good yield. This compound was then converted into the sodium salt 59 via ion exchange on resin columns Dowex H⁺ and subsequently Dowex Na⁺ in excellent yields. As expected, only one product was obtained since this disaccharide lacks a free anomeric hydroxyl group.

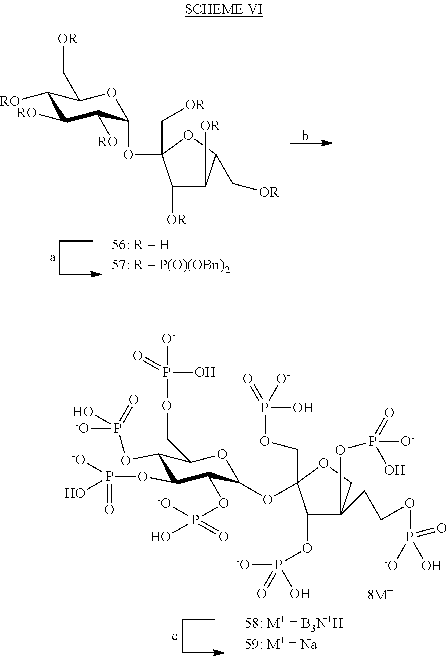

Synthesis of Octakis Phosphorylated Disaccharides

Scheme VII shows the synthesis of the pyrophosphates 60-62 55 (62 is II-1) of the tetrakis phosphorylated methyl glycosides of glucose 39 and mannose 40 derivatives. The reagents and conditions used, with reference to Scheme VII, included: a) DCC, MeCN, 82° C. b) Dowex H⁺, H₂O then Dowex Na⁺, H₂O. For the synthesis of PPs, two vicinal phosphates, or an even number of phosphates, all in pairs, are required. Condensation reactions of phosphates, in particular in the conversion of IHP into ITPP, were usually performed in pyridine, using the IHP pyridinium salt. To avoid the use of a rather large amount of an unpleasant and toxic solvent, especially when multigram synthesis is required, a modified coupling reaction for the synthesis of ITPP, via its triethylammonium salt was developed. The method involved dissolution of the IHP triethylammonium salt in a mixture of MeCN/H₂O (ratio of 2/1) and heating the solution at reflux in the presence of excess N,N″-dicyclohexyl carbodiimide (DCC). The combination of these solvents was used as they are miscible and both polar triethylammonium salt and lipophilic DCC is soluble in the mixture. For other solvent ratios, either DCC or the salt may remain partially undissolved. In line with these results, the formation of the triethylammonium salts was also implemented for the present synthesis of cyclic PP derivatives.

Complete and clean transformation to cyclic PPs could be achieved from tetrakis phosphorylated hexopyranoses having the remaining hydroxyl group masked, i.e., the anomeric one in the case of the 2,3,4,6-tetrakis phosphorylated derivatives. Although substrates 39 and 40 are structurally similar to IHP, when the reactions were performed under the same conditions applied for ITPP, products with two PPs in a row were not obtained in a regio-controlled way.

To evaluate if water was responsible for this failure to prepare these PPs, the reactions were conducted in neat MeCN, with the possibility that the triethylammonium salt, insoluble at room temperature, would be solubilized in the refluxing solvent. Indeed, when glucose salt derivative 39 was dissolved in refluxing MeCN in the presence of excess DCC, the bis PP 60 was formed in 95% yield. The same result was obtained in the case of mannose salt 40. The ³¹P NMR spectrum of the crude reaction mixture showed complete consumption of the starting phosphate and the exclusive formation of 61. Two pairs of doublets, with coupling constants of 25.5 and 22.0 Hz respectively, appeared, indicating two AB systems that correspond to the eight and seven membered cyclic PPs. The same pattern was observed in the spectra of glucose derivative 60 with coupling constants of 24.9 and 17.9 Hz, respectively. The latter was easily purified by filtration to remove the formed dicyclohexyl urea (DCU) from the resulting aqueous solution. Then, it was transformed into the corresponding sodium salt 62 by ion exchange. In contrast, mannose bis-PP 61 was found to decompose during the aqueous work up. While not wishing to be bound by theory, this may be due to instability of the cis seven-membered pyrophosphate of this compound.

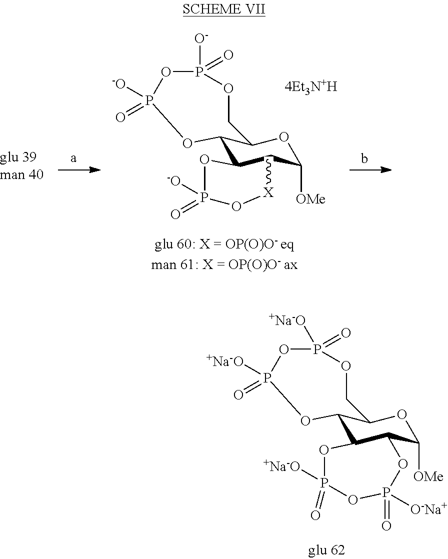

### Example 2

### Mediation of Oxygen Release/Delivery

Selected compounds were tested for their abilities to allosterically affect hemoglobin. The compounds studied were:

| | Compound | |
|---|---|---|
| Name | Number | Structure |
| 1-O-methyl-α-glucose 2,3,4-trisphosphate | 8; I-1 | 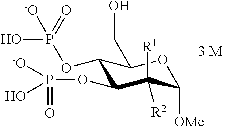 |
| | | glu: R¹ = H, R² = OP(O)(OH)O⁻ |
| | | M⁺ = Na⁺ |
| 1-O-methyl-α-mannose 2,3,4-trisphosphate | 10; I-2 | 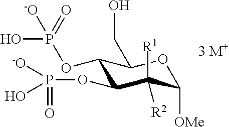 |
| | | man: R¹ = OP(O)(OH)O⁻, R² = H |
| | | M⁺ = Na⁺ |
| α-glucose 1,2,3,4- tetrakisphosphate | 29; I-3 | 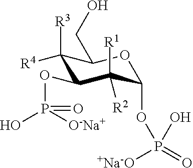 |
| | | R¹, R³ = H, R², R⁴ = OP(O)(OH)(ONa) |
| β-glucose 1,2,3,4- tetrakisphosphate | 30; I-4 | 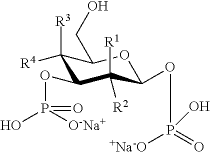 |
| | | R¹, R³ = H, R², R⁴ = OP(O)(OH)(ONa) |
| α-mannose 1,2,3,4- tetrakisphosphate | 31; I-5 | 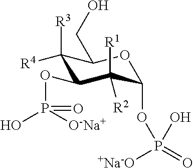 |
| | | R², R³ = H, R¹, R⁴ = OP(O)(OH)(ONa) |
| β-mannose 1,2,3,4- tetrakisphosphate | 32; I-6 | 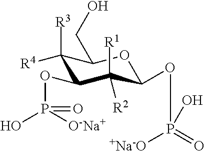 |
| | | R², R³ = H, R¹, R⁴ = OP(O)(OH)(ONa) |
| α-galactose 1,2,3,4- tetrakisphosphate | 33; I-7 | 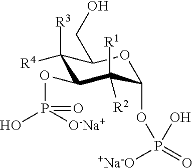 |
| | | R¹, R⁴ = H, R², R³ = OP(O)(OH)(ONa) |
| β-galactose 1,2,3,4- tetrakisphosphate | 34; I-8 | 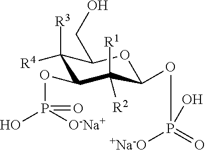 |
| | | R¹, R⁴ = H, R², R³ = OP(O)(OH)(ONa) |
| 1-O-methyl-α-glucose tetrakisphosphate | 41; I-9 | 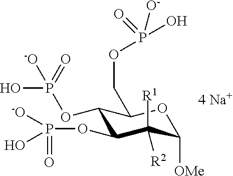 |
| | | R¹ = H, R² = OP(O)(OH)O⁻ |
| 1-O-methyl-α-mannose tetrakisphosphate | 42; I-10 | 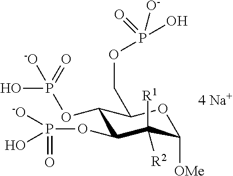 |
| | | R¹ = OP(O)(OH)O⁻, R² = H |
| α-glucose pentakisphosphate | 47; I-11 | 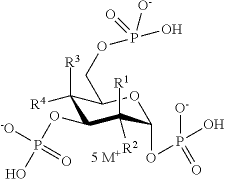 |
| | | glu: R¹, R³ = H, R², R⁴ = OP(O)(OH)O⁻ |
| | | M⁺ = Na⁺ |
| α-mannose pentakisphosphate | 49; I-12 | 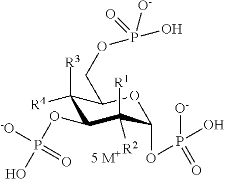 |
| | | man: R², R³ = H, R¹, R⁴ = OP(O)(OH)O⁻ |
| | | M⁺ = Na⁺ |
| α-galactose pentakisphosphate | 51; I-13 | 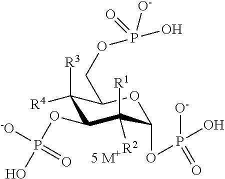 |
| | | gal: R¹, R⁴ = H, R², R³ = OP(O)(OH)O⁻ |
| | | M⁺ = Na⁺ |
| lactose octakisphosphate | 55; I-14 | 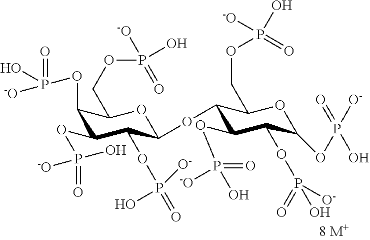 |
| | | M⁺ = Na⁺ |
| sucrose octakisphosphate | 59; I-15 | 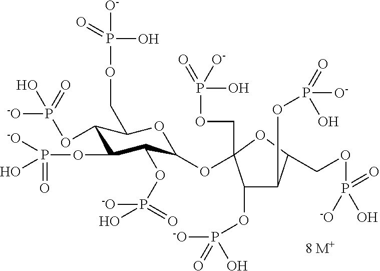 |
| | | M⁺ = Na⁺ |
| 1-O-methyl-α-glucose bispyrophosphate | 62; II-1 | 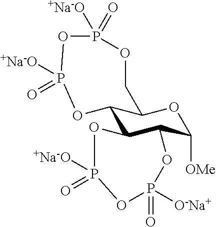 |

Preparation of Stripped Hemoglobin:

Human blood was withdrawn from non-smoking healthy volunteers (CDD) in heparinized microtubes and treated according to the procedure described by Riggs. See A. Riggs, Methods Enzymol. 1981, 76, 5. Red blood cells were washed three times with 0.85% saline and lysed by the addition of 1 volume of purified water per volume of packed red blood cells. Hemolysate was kept cold and passed through a Sephadex G-25 column (1.0×20 cm) equilibrated with 0.1 M NaCl with 10⁻⁵ M EDTA pH 7.5, to remove BPG. The concentration of oxy-Hb was assessed by UV-Vis spectrophotometry (ε=58400 M⁻¹ cm⁻¹ at 577 nm per oxy-Hb tetramer).

General Procedure for Oxygen Equilibration Curves Measurements:

Solutions of the test compounds (supra, 100 mM) were prepared in purified water and the pH was adjusted to 7.0-7.4 prior to incubations with stripped Hb in a molar ratio of 20:1. The mixtures were diluted in 3 ml of TES-saline buffer (30 mM TES, 140 mM saline, pH 7.4) and oxygen equilibrium curves were measured using a Hemox Analyzer apparatus (TCS Scientific Corp., USA). The P₅₀ values and Hill coefficients were calculated by linear regression analysis from data points obtained between 40 and 60% oxygen saturation.

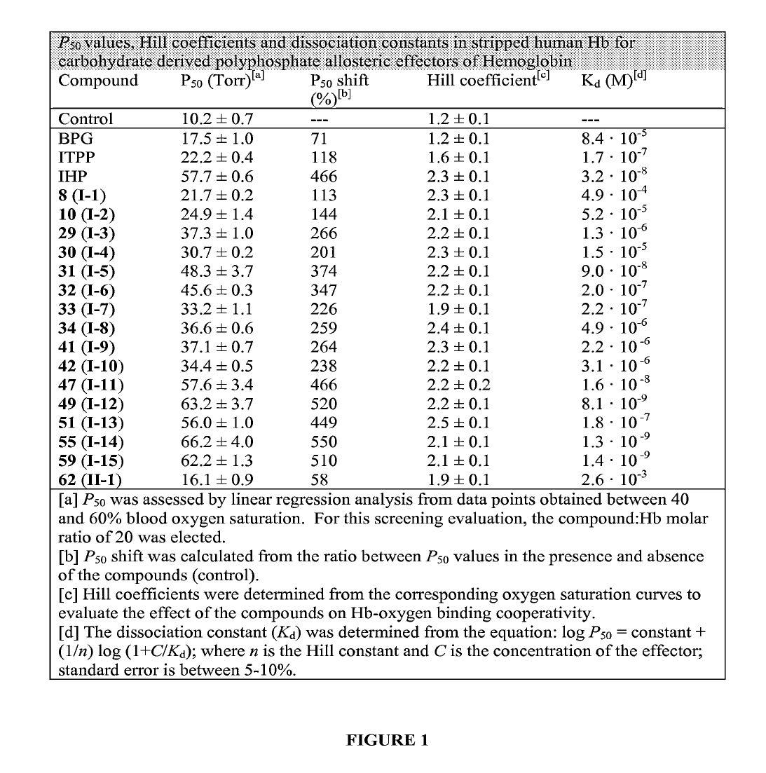

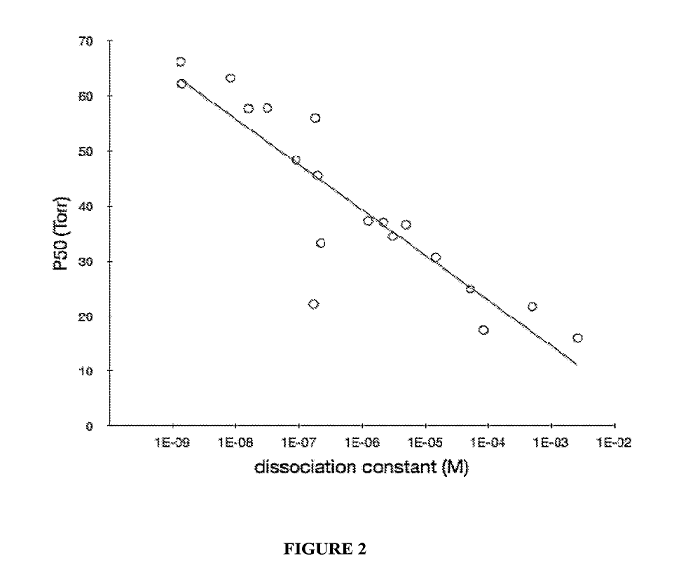

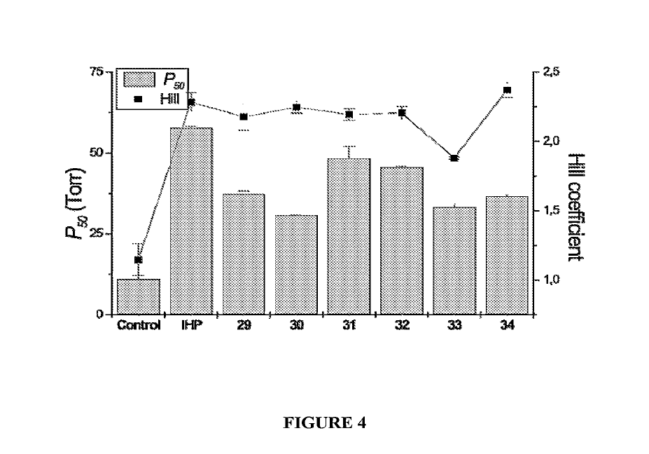

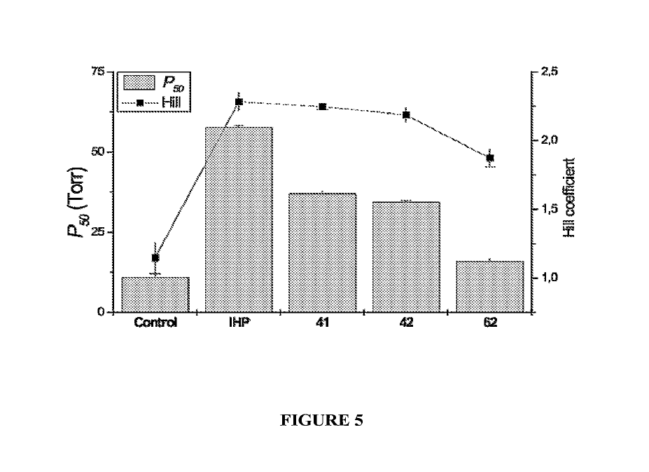

All of the studied compounds were able to shift the Hb oxygenation curves, from 58% up to 550% (FIG. 1). The relationship between binding to Hb and oxygen release is illustrated in FIG. 2. Without wishing to be bound by theory, the ability of the compounds to lower the Hb affinity to oxygen may be directly related to their number of negative charges; that is, the greater the number of phosphates the higher the P₅₀ value. For instance, octakisphosphosphate carbohydrates 55 (I-14) (550%) and 59 (I-15) (550%) were more effective than trisphosphate compounds 8 (I-1) (113%) and 10 (I-2) (144%). The same trend was observed for the known compounds: BPG<ITPP<IHP (FIGS. 1-7). These observations are in agreement with the fact that the allosteric pocket of Hb is particularly rich in positively charged amino acid residues, located on the β subunits at the entrance to the Hb central cavity,^{[2]} which would favor the tight docking of polyanions by electrostactic interaction. As a result, the number of negative charges being directly proportional to the strength of electrostactic interaction, a larger number would be expected to induce the formation of a tighter Hb-effector complex. Indeed, the direct correlation between the affinity of the present compounds towards Hb and their ability to induce oxygen release is consistent with the above. FIG. 2 shows that the larger the P₅₀ shift, the higher the affinity of the compounds for Hb, which is directly linked to their number of charges, i.e., phosphate groups, and results in a tight electrostactic docking of the compounds into the allosteric pocket of Hb.

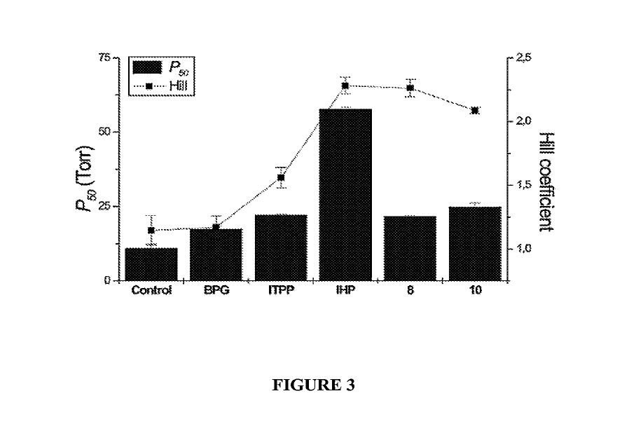

With respect to the specificities observed within the distinct series of hexopyranose polyphosphate compounds, mannose derivatives (10 (I-2), 31 (I-5), 32 (I-6), and 49 (I-12)) were generally more effective than their corresponding glucose (8 (I-1), 29 (I-3), 30 (I-4), and 47 (I-11)) and galactose (33 (I-7), 34 (I-8), and 51 (I-13)) analogs (FIGS. 3-6). One exception to this behavior was the compound 42 (I-10), 1-O-methyl-α-mannose tetrakisphosphate, which was less effective than the other mannose tetrakisphosphates derivatives (31 (I-5) and 32 (I-6)). Such a reduction in effectiveness, could be due, without wishing to be bound by theory, to the absence of the C₁-phosphate group in 42 (I-10), which may be important for molecular recognition of the mannose polyphosphates.

Among the glucose series, without wishing to be bound by theory, the absence of either C₁ or C₆ phosphate groups may lead to the same effect within the α anomers, i.e. compounds 29 (I-3) and 41 (I-9) (266 and 264%, respectively); however, the corresponding β anomer of glucose 1,2,3,4-tetrakisphosphate (30 (I-4)) was less effective (201%). Conversely, within the galactose series, the β anomer of galactose 1,2,3,4-tetrakisphosphate (34 (I-8)) is more effective than its alpha anomer 33 (I-7) (259 versus 226%, respectively). Furthermore, compounds α-glucose 1,2,3,4-tetrakisphosphate (31 (I-5)) and β-galactose 1,2,3,4-tetrakisphosphate (32 (I-6)) showed similar effects on oxygen release, probably due, but not wishing to be bound by theory, to conformational similarities in the presentation of 1 axial and 3 equatorial (1ax-3eq) phosphate groups in both cases.

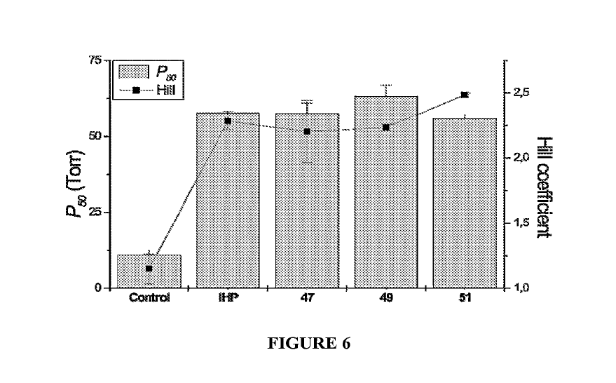

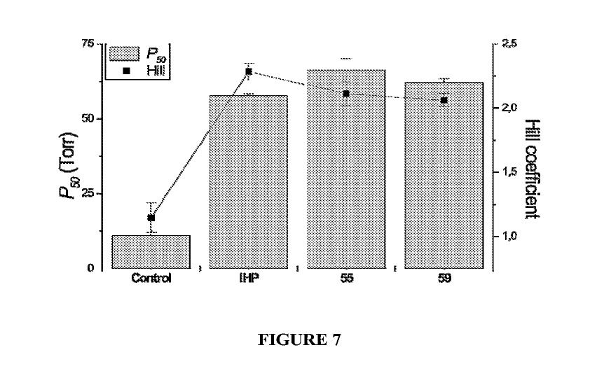

Octakisphosphate disaccharides 55 (I-14; 550%) and 59 (I-15; 510%) were able to lower Hb affinity to oxygen in a moderately higher fashion than IHP itself (466%, FIG. 7), in comparison to monosaccharides 47 (I-11; 466%), 49 (I-12; 520%) and 51 (I-13; 449%). The fact that the latter compounds, which contain only five phosphate groups, nevertheless induce allosteric effects comparable to that of IHP (FIG. 6) might, but not wishing to be bound by theory, be related to access to the allosteric binding site. This suggests that for the disaccharides 55 (I-14) and 59 (I-15), which are more voluminous than the monosaccharides 47 (I-11), 49 (I-12), and 51 (I-13), steric effects may also play a role (perhaps secondary to electrostatic factors) regarding the access to the binding site.

As a result, the observed differences in activity for disaccharides 55 (I-14) and 59 (I-15) may, without wishing to be bound by theory, be related to the docking mode of these compounds, as they are able to bind to Hb through the interaction with either the hexopyranose or pentofuranose subunits. As a result, the docking of these compounds to Hb is statistically increased by their dual binding mode. Furthermore, this may explain why there is only a slight difference in activity between lactose 55 (I-14) and sucrose 59 (I-15), the sugar scaffold playing apparently a minor role on selectivity for phosphorylated disaccharides.

### Example 3

### Inhibition of PI3K Activity

Several compounds were tested for activity against class I PI3K. The assay was conducted using the HTRF Assay Platform (Reaction Biology Corporation (RBC), Malvern, Pa.). In this assay, PIP3 product is detected by displacement of biotin-PIP3 from an energy transfer complex consisting of Europium labeled anti-GST monoclonal antibody, a GST-tagged pleckstrin homology (PH) domain, biotinylated PIP3 and Streptavidin-Allophycocyanin (APC). Excitation of Europium in the complex results in an energy transfer to the APC and a fluorescent emission at 665 nm. The PIP3 product formed by PI3-Kinase (h) activity displaces biotin-PIP3 from the complex resulting in a loss of energy transfer and thus a decrease in signal.

In short, the substrate (10 uM PIP₂ substrate (PI(4,5)P₂)) was prepared in freshly made Reaction Buffer and the kinase was delivered to the solution with gentle mixing. The compound was then added into the kinase reaction mixture manually, and allowed to incubate for 10 minutes at room temperature. After this incubation, ATP (10 uM) was added into the reaction mixture to initiate the reaction and the reaction progressed for 30 min at 30° C. The reaction was quenched with Stop Solution; the Detection Mixture was added and allowed to incubate overnight. The next day, the reactions were measured by homogeneous time resolved fluorescence: Ex=320 nm, ratio of Em=615 nm and Em=665 nm.

The following enzymes were tested:

Human PI3Kα (p110α/p85α): Complex of N-terminal GST-tagged recombinant full-length human p110α (GenBank Accession No. U79143), and recombinant full length, human p85α (no tag) (GenBank Accession No. XM_{—}043865). Coexpressed in a Baculovirus infected Sf9 cell expression system. p110α MW=155 kDa, p85α MW=83.5 kDa.

Human PI3Kβ (p110β/p85α): Complex of N-terminal 6His-tagged recombinant full-length human p110β (GenBank Accession No. NM_{—}006219), and recombinant full length, human p85α (no tag) (GenBank Accession No. XM_{—}043865). Coexpressed in a Baculovirus infected Sf21 cell expression system. p110β MW=124 kDa, p85α MW=83.7 kDa.

Human PI3Kγ (p120γ): (GenBank Accession No. AF327656), full length with N-terminal His tag, expressed in a Baculovirus infected Sf9 cell expression system. MW=131 kDa.

Human PI3Kδ (p110δ/p85α): Complex of N-terminal GST tagged recombinant full-length human p110δ (GenBank Accession No. NM_{—}005026), and recombinant full length, human p85α (GenBank Accession No. XM_{—}043865). Coexpressed in a Baculovirus infected Sf9 cell expression system. p110δ MW=146 kDa, p85α MW=83.5 kDa.

As shown in FIG. 8, several compounds showed activity against PI3K. The % inhibition of PI3Kα, PI3Kβ, PI3Kγ, PI3Kδ is shown by a scoring system whereby (+++) is the highest inhibitory effect and (−) is the lowest.

### INCORPORATION BY REFERENCE

All patents and publications referenced herein are hereby incorporated by reference in their entireties.

1. A pharmaceutical composition comprising a compound that is a polyphosphate or pyrophosphate derivative of a mono-, di- or oligosaccharide containing a pyranose or furanose unit, or a pharmaceutically acceptable salt thereof.

2. The pharmaceutical composition of claim 1, wherein the compound is a phosphate or polyphosphate derivative of glucose, mannose, or galactose.

3. The pharmaceutical composition of claim 2, wherein the pyranose is part of an oligosaccharide comprising from 2 to 4 monosaccharide units.

4. The pharmaceutical composition of claim 3, wherein the oligosaccharide is a phosphate or polyphosphate derivative of sucrose or lactose.

5. The pharmaceutical composition of claim 1, wherein the compound comprises from 2 to about 10 phosphate or polyphosphate groups or pyrophosphate groups.

6. (canceled)

7. (canceled)

8. The pharmaceutical composition of claim 1, wherein the pyranose further comprises a derivatized hydroxyl selected from alkoxy (—OR) or acyloxy (—OCOR), where R is selected from alkyl, aryl, acyl, aralkyl, alkenyl, alkynyl, heterocyclyl, polycyclyl, carbocycle, amino, acylamino, amido, alkylthio, carbonyl, sulfonate, alkoxyl, sulfonyl, or sulfoxido.

9. (canceled)

10. (canceled)

11. (canceled)

12. A compound of Formula I 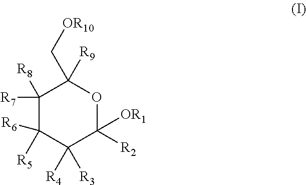
wherein:
R₁ and R₁₀ are independently H, C₁-C₆ alkyl, C₁-C₆ haloalkyl, aryl C₁-C₆ alkyl, phosphate, polyphosphate, 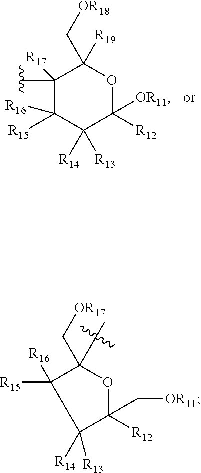
R₂ is H;
R₃, R₄, R₅, R₆, R₇, R₈, R₉, and R₁₀ are independently H, C₁-C₆ alkyl, C₁-C₆ alkenyl, C₁-C₆ alkynyl, C₁-C₆ haloalkyl, aryl C₁-C₆ alkyl, phosphate, polyphosphate; and
R₁₁, R₁₂, R₁₃, R₁₄, R₁₅, R₁₆, R₁₇, R₁₈, and R₁₉ are independently H, OH, phosphate, or polyphosphate; wherein at least one of R1 to R10 are phosphate or polyphosphate;
or a pharmaceutical acceptable salt, stereoisomer, anomer, solvate, and hydrate thereof.

13. The compound according to claim 12, wherein the compound is: 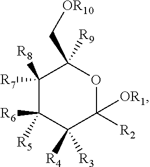
wherein at least one, two, or three of R₃, R₆, and R₇ are phosphate or polyphosphate.

14. (canceled)

15. (canceled)

16. (canceled)

17. (canceled)

18. The compound according to claim 12, wherein the compound is 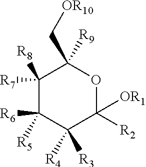
and wherein R₃, R₆, and R₇ are phosphate.

19. (canceled)

20. (canceled)

21. (canceled)

22. (canceled)

23. The compound according to claim 12, wherein the compound is 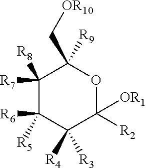
and wherein R₃, R₆, and R₇ are phosphate.

24. (canceled)

25. (canceled)

26. (canceled)

27. (canceled)

28. The compound according to claim 12, wherein the compound is a D-isomer or L-isomer.

29. (canceled)

30. The compound according to claim 12, wherein the anomer is in the α form or β form.

31. (canceled)

32. The compound according to claim 12, wherein the compound is selected from the group consisting of:
1-O-methyl-α-glucose 2,3,4-trisphosphate (I-1);
1-O-methyl-α-mannose 2,3,4-trisphosphate (I-2);
α-glucose 1,2,3,4-tetrakisphosphate (I-3);
β-glucose 1,2,3,4-tetrakisphosphate (I-4);
α-mannose 1,2,3,4-tetrakisphosphate (I-5);
β-mannose 1,2,3,4-tetrakisphosphate (I-6);
α-galactose 1,2,3,4-tetrakisphosphate (I-7);
β-galactose 1,2,3,4-tetrakisphosphate (I-8);
1-O-methyl-α-glucose tetrakisphosphate (I-9);
1-O-methyl-α-mannose tetrakisphosphate (I-10);
α-glucose pentakisphosphate (I-11);
α-mannose pentakisphosphate (I-12);
α-galactose pentakisphosphate (I-13);
lactose octakisphosphate (I-14); and
sucrose octakisphosphate (I-15).

33. A compound of Formula II: 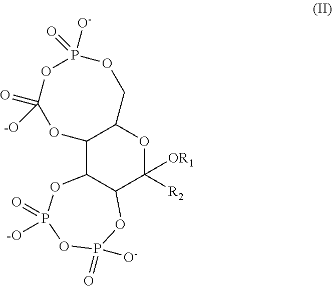
wherein:
R₁ is H, C₁-C₆ alkyl, C₁-C₆ alkenyl, C₁-C₆ alkynyl, C₁-C₆ haloalkyl, aryl C₁-C₆ alkyl, phosphate, or polyphosphate; and
R₂ is H
or a pharmaceutical acceptable salt, stereoisomer, anomer, solvate, and hydrate thereof.

34. The compound according to claim 33, wherein the compound is 1-O-methyl-α-glucose bispyrophosphate (II-1).

35. A method of treating cancer, comprising administering to a subject in need thereof a pharmaceutical composition or compound of claim 1.

36. The method of claim 35, wherein the cancer is a breast cancer, prostate cancer, renal cell cancer, brain cancer, ovarian cancer, colon cancer, bladder cancer, pancreatic cancer, stomach cancer, esophageal cancer, cutaneous melanoma, liver cancer, lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, lip and oral cancer, skin cancer, leukemia or multiple myeloma.

37. The method of claim 35, further comprising administering to the subject a therapeutically effective amount of a chemotherapeutic agent and/or radiation therapy.

38. A method of treating a cardiovascular disease comprising administering to a subject in need thereof a pharmaceutical composition or compound of claim 1, wherein the cardiovascular disease is a coronary infarction, a pulmonary disease, congestive heart failure, a myocardial infarction, a peripheral vascular disease, stroke, an intermittent claudication, or arteriosclerosis.

39. (canceled)

40. (canceled)

41. A method of enhancing oxygen delivery to a tissue or organ of a mammal, comprising administering to a subject in need thereof a pharmaceutical composition or compound of claim 1.
